(19) United States
(12) Patent Application Publication
(10) Pub. No.: US 2005/0002888 A1
(43) Pub. Date: Jan. 6, 2005

(54) Water-in-silicone emulsions

(75) Inventors: Andreas Bleckmann, Ahrensburg (DE); Silke Kohlhase, Hamburg (DE); Rainer Kropke, Schenefeld (DE); Inka Longree, Hamburg (DE); Boris Syskowski, Hamburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/849,805

(22) Filed: May 21, 2004

Related U.S. Application Data
(63) Continuation of application No. PCT/EP02/13053, filed on Nov. 21, 2002.

(30) Foreign Application Priority Data
Nov. 23, 2001 (DE) 101 57 542.4

(51) Int. Cl. A61K 7/06; A61K 7/11

(52) U.S. Cl. 424/70.12; 424/70.13

(57) ABSTRACT

Water-in-silicone emulsion comprising a) an aqueous phase, b) a lipid phase having a silicone content of at least 50% by weight, based on the lipid phase, c) emulsifiers, d) starch and/or starch derivatives, besides, if appropriate, further cosmetic and/or dermatological active ingredients, excipients and additives.

The present invention relates to water-in-silicone emulsions comprising starch and/or starch derivatives, and to their use.

Emulsions are understood in general as meaning heterogeneous systems which consist of two liquids which are immiscible or only limitedly miscible with one another, which are customarily designated as phases. In an emulsion, one of the two liquids is dispersed in the other liquid in the form of very fine droplets.

If the two liquids are water and oil and oil droplets are present in water in finely dispersed form, the emulsion is an oil-in-water emulsion (O/W emulsion, e.g. milk). The basic character of an O/w emulsion is shaped by the water. In a water-in-oil emulsion (W/O emulsion, e.g. butter), it is the opposite principle, the basic character being determined here by the oil.

Beside the emulsions which consist of water and oils based on carbon, there are also those in which silicone oil are employed as the lipid phase. These are used, for example, in cosmetics in order to impart an elegant, silky skin sensation to the preparations. Relatively large amounts of humectants (moisturizers), in particular glycerol, can furthermore be incorporated in these emulsions without the preparation having a sticky sensory function.

Cosmetic and/or dermatological water-in-silicone emulsions must, however, customarily be stabilized with waxes or pigments as stabilizers in a relatively high concentration. These have the disadvantage that they cannot be dispersed on the skin particularly well and, after application, leave behind a sticky residue on the skin. With relatively high proportions of silicone oils in the emulsion, a large quantity of residue accordingly remains behind on the skin and and on application a fatty-oily skin sensation is obtained.

It was therefore the object of the present invention to remedy the disadvantages of the prior art and and to develop water-in-silicone emulsions having a pleasant skin sensation and which are stable long-term.

Surprisingly, the object is achieved by water-in-silicone emulsions comprising
a) an aqueous phase,
b) a lipid phase having a silicone content of at least 50% by weight, based on the lipid phase,
c) emulsifiers,
d) starch and/or starch derivatives,
   beside, if appropriate, further cosmetic and/or dermatological active ingredients, excipients and additives.

On cosmetic and/or dermatological application thereof, these emulsions produce a silky light skin sensation in combination with a pleasantly dry, powdery residue on the skin.

Moreover, they are stable for over 30 months on storage at room temperature. These positive characteristics are retained even if there is an increased proportion of humectants in the emulsion, such as, for example, in the case of the use of over 10% by weight of glycerol, and on addition of up to 50% by weight of nonpolar lipids (e.g. mineral oils, petroleum jelly, dicaprylyl ether), based on the total weight of the lipid phase.

The water-in-silicone emulsions according to the invention contain starch and/or starch derivatives in an amount of from 0.1 to 20% by weight and particularly preferably from 0.5 to 10% by weight, based on the total weight of the emulsion.

The starches and/or starch derivatives preferred according to the invention are dimethylimidazolidone rice starch [e.g. Rice NS from Chemag], aluminum starch octenylsuccinate [Dry Flo PC from National Starch] and/or tapioca [=manihot starch, e.g. Tapioca Pure/Tapioca Starch from National Starch].

For the water-in-silicone emulsions according to the invention, all emulsifiers known for this type of emulsion can be employed. Particularly preferred water-in-silicone emulsifiers according to the invention are in this case cetyldimethicone copolyol and laurylmethicone copolyol [e.g. ABIL EM 90 (Goldschmidt), DC5200 Formulation Aid (Dow Corning)], and any desired mixtures of the two emulsifiers.

Very particularly advantageous according to the invention is the combination of dimethyl-imidazolidone rice starch [e.g. Rice NS from Chemag] as the starch derivative and alkyldimethicone copolyol [e.g. ABIL EM 90 (Goldschmidt), DC5200 Formulation Aid (Dow Corning)] as the emulsifier, which make possible both flowable lotions and soft creams of low consistency and high skincare capacity, in particular with respect to moisturizing the skin and smoothing the skin.

The lipid phase of the water-in-silicone emulsions according to the invention can advantageously be chosen from the group consisting of the polar oils, for example from the group consisting of the lecithins and the fatty acid triglycerides, especially the triglycerides of saturated and/or unsaturated, branched and/or unbranched alkane-carboxylic acids of a chain length of 8 to 24, in particular 12 to 18 carbon atoms. The fatty acid triglycerides can, for example, advantageously be chosen from the group consisting of the synthetic, semisynthetic and natural oils, such as, for example, coconut glyceride, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil and suchlike.

Furthermore advantageous according to the invention are, for example, natural waxes of animal and vegetable origin, such as, for example, beeswax and other insect waxes, and also berry wax, shea butter and/or lanolin (wool wax).

Within the meaning of the present invention, further advantageous polar oil components can furthermore be chosen from the group consisting of the esters of saturated and/or unsaturated, branched and/or unbranched alkanecarboxylic acids of a chain length of 3 to 30 carbon atoms and saturated and/or unsaturated, branched and/or unbranched alcohols of a chain length of 3 to 30 carbon atoms, and from the group consisting of the esters of aromatic carboxylic acids and saturated and/or unsaturated, branched and/or unbranched alcohols of a chain length of 3 to 30 carbon atoms. Such ester oils can then advantageously be chosen from the group consisting of octyl palmitate, octyl cocoate, octyl isostearate, octyldodeceyl myristate, octyldodecanol, cetearyl isononanoate, isopropyl myristate, isopropyl palmitate, isopropyl stearate, isopropyl oleate, n-butyl stearate, n-hexyl laurate, n-decyl oleate, isooctyl stearate, isononyl stearate, isononyl -isononanoate, 2-ethylhexyl palmitate, 2-ethylhexyl laurate, 2-hexyldecyl stearate, 2-octyl-dodecyl palmitate, stearyl heptanoate, oleyl oleate, oleyl erucate, erucyl oleate, erucyl erucate, tridecyl stearate, tridecyl trimellitate, and also synthetic, semisynthetic and natural mixtures of such esters, such as, for example, jojoba oil.

The lipid phase can furthermore be advantageously chosen from the group consisting of the dialkyl ethers and dialkyl carbonates, e.g. dicaprylyl ether (Cetiol OE) and/or dicaprylyl carbonate are advantageous, for example that obtainable under the trade name Cetiol CC from Cognis.

It is furthermore advantageous the oil component(s) from the group consisting of isoeicosane, neopentyl glycol diheptanoate, propylene glycol dicaprylate/dicaprate, caprylic/capric/diglyceryl succinate, butylene glycol dicaprylate/dicaprate, coconut glycerides (e.g. Myritol®) 331 from Henkel), C₁₂₋₁₃-alkyl lactate, di-Cl₂₁₃-alkyl tartrate, tri-isostearin, dipentaerythrityl hexacaprylate/hexacaprate, propylene glycol monoisostearate, tricaprylin, dimethyl isosorbide. It is particularly advantageous if the lipid phase of the formulations according to the invention contains C₁₂₋₁₅-alkyl benzoate or consists completely of this.

Advantageous oil components are furthermore, for example, butyl octylsalicylate (for example that obtainable under the trade name Hallbrite BHB from CP Hall), hexadecyl benzoate and butyl octylbenzoate and mixtures thereof (Hallstar AB) and/or diethylhexyl naphthalate (Hallbrite TQ from CP Hall or Corapan®TQ from Haarmann & Reimer).

Any desired mixtures of such oil and wax components can also be employed advantageously within the meaning of the present invention.

The lipid phase can, according to the invention, contain the polar oil components in a concentration of up to 40% by weight based on the total weight of the lipid phase.

Furthermore, the lipid phase can likewise advantageously also contain nonpolar oils, for example those which are chosen from the group consisting of the branched and un-branched hydrocarbons and hydrocarbon waxes, in particular mineral oil, petroleum jelly, (petrolatum), paraffin oil, squalane and squalene, polyolefins, hydrogenated polyisobutenes and isohexadecane. Among the polyolefins, polydecenes are the preferred substances.

The nonpolar oil components can be present according to the invention in a concentration of up to 50% by weight based on the total weight of the lipid phase in the emulsions according to the invention.

The lipid phase according to the invention further contains silicone oils. It can contain cyclic or linear silicone oils or consist completely of such oils, it being preferred, however, aside from the silicone oil or the silicone oils to use an additional content of other oil-phase components.

Silicone oils are high molecular weight synthetic polymeric compounds, in which silicon atoms are linked in a chainlike and/or networklike manner via oxygen atoms and the remaining valencies of the silicon are saturated by hydrocarbon radicals (usually methyl groups, more rarely ethyl, propyl, phenyl groups and the like). Systematically, the silicone oils are designated as polyorganosiloxanes. The methyl-substituted polyorganosiloxanes, which are quantitatively the most important compounds of this group and are distinguished by the following structural formula 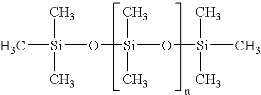
are also designated as Polydimethylsiloxane and Dimethicone (INCI). Dimethicones exist in various chain lengths and with various molecular weights.

Advantageous polyorganosiloxanes within the meaning of the present invention are, for example, dimethylpolysiloxane [poly(dimethylsiloxane)], which are obtainable from Goldschmidt, for example, under the trade names Abil 10 to 10 000. Furthermore advantageous are phenylmethylpolysiloxane (INCI: Phenyl Dimethicone, Phenyl Tri-methicone), cyclic silicones (octamethylcyclotetrasiloxane and decamethylcyclopenta-siloxane), which are also designated according to INCI as Cyclomethicone, amino-modified silicones (INCI: Amodimethicone) and silicone waxes, e.g. polysiloxane-poly-alkylene copolymers (INCI: Stearyl Dimethicone and Cetyl Dimethicone) and dialkoxydimethylpolysiloxanes (Stearoxy Dimethicone and Behenoxy Stearyl Dimethicone), which are obtainable from Goldschmidt as various Abil wax types. However, other silicone oils can also be used advantageously within the meaning of the present invention, for example cetyldimethicone, hexamethylcyclotrisiloxane, polydimethylsiloxane, poly(me-thylphenylsiloxane).

Particularly preferred silicones according to the invention are dimethicone and cyclomethicone.

The silicone content of the lipid phase can, according to the invention, be 50 to 100% by weight and particularly preferably from 75 to 95% by weight, in each case based on the total weight of the lipid phase.

The aqueous phase of the emulsions according to the invention can advantageously contain customary cosmetic excipients, such as, for example, alcohols, in particular those of low carbon number, preferably ethanol and/or isopropanol, diols or polyols of low carbon number, and their ethers, preferably propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes, dihydoxyacetone, and in particular one or more thickening agents, which can advantageously be chosen from the group consisting of silica, aluminum silicates, polysaccharides and their derivatives, e.g. hyaluronic acid, xanthan gum, hydroxypropylmethylcellulose, particularly advantageously from the group consisting of the polyacrylates, preferably a polyacrylate from the group consisting of the “carbopols”, for example carbopols of the types 980, 981, 1382, 2984, 5984, in each case individually or in combination.

Furthermore advantageous are copolymers of C₁₀₋₃₀-alkyl acrylates and one or more monomers of acrylic acid, methacrylic acid or their esters.

Compounds are advantageous which carry the INCI name “Acrylates/C₁₀₋₃₀ Alkyl Acrylate Crosspolymer”. Those obtainable under the trade names Pemulen TR1 and Pemulen TR2 from the B. F. Goodrich Company are particularly advantageous.

Compounds are advantageous which carry the INCI name Ammonium acryloyldimethyltaurate/Vinylpyrrolidone copolymer.

According to the invention, the ammonium acryloyldimethyltaurate/vinylpyrrolidone copolymer(s) advantageously have the empirical formula [C₇H₁₆N₂SO₄]ₙ [C₆H₉NO]ₘ, corresponding to a random structure as follows

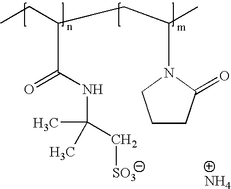

Preferred species within the meaning of the present invention are filed in Chemical Abstracts under the registry numbers 58374-69-9, 13162-05-5 and 88-12-0 and are obtainable under the trade name Aristoflex® AVC of Clariant GmbH.

Furthermore advantageous are copolymers/crosspolymers comprising Acryloyldimethyl Taurate, such as, for example, Simugel® EG or Simugel® EG from Seppic S.A.

Further thickening agents which can be used advantageously according to the invention are also anionic polyurethanes which are soluble or dispersible in water. Those advantageous within the meaning of the present invention are, for example, polyurethane-1 and/or polyurethane-4.

Particularly advantageous polyurethanes within the meaning of the present invention are the types obtainable under the trade name Avalure™ UR from the B. F. Goodrich Company, such as, for example, Avalure™ UR 445, Avalure™ UR 450 and the like. Furthermore advantageous within the meaning of the invention is also the polyurethane obtainable under the trade name Luviset Pur from BASF.

Humectants (moisturizers) can preferably also be used. Moisturizers are designated as substances or substance mixtures which impart to cosmetic or dermatological preparations the property, after application to or dispersion on the skin surface, of reducing the release of moisture from the horny layer (also called transepidermal water loss (TEWL)) and/or positively influencing the hydration of the horny layer.

Advantageous moisturizers within the meaning of the present invention are, for example, glycerol, lactic acid and/or lactates, in particular sodium lactate, butylene glycol, propylene glycol, biosaccaride gum-1, Glycine soja, ethylhexyloxyglycerol, pyrrolidone-carboxylic acid and urea. Furthermore, it is particularly advantageous to use polymeric moisturizers from the group consisting of the polysaccharides which are water-soluble and/or swellable in water and/or gellable with the aid of water. For example, hyaluronic acid, chitosan and/or a fucose-rich polysaccharide, which is filed in Chemical Abstracts under the registry number 178463-23-5 and, for example, is obtainable under the name Fucogel®1000 from SOLABIA S.A., are particularly advantageous.

It is advantageous according to the invention if the water-in-silicone emulsion contains humectants in a concentration of 5 to 25% by weight and particularly preferably in a concentration of 7.5 to 15% by weight, in each case based on the total weight of the emulsion.

The ratio of aqueous phase to lipid phase in the water-in-silicone emulsions according to the invention is advantageously 65:35 to 80:20, the ratio from 70:30 to 75:25 (aqueous phase to lipid phase) being particularly preferred.

The water-in-silicone emulsions according to the invention can contain cosmetic excipients, such as are customarily used in such preparations, e.g. preservatives, preservation aids, bactericides, perfumes, substances for preventing foaming, colorants, pigments which have a coloring action, thickening agents, moisturizing and/or humectant substances, fillers which improve the skin sensation, fats, oils, waxes or other customary constituents of a cosmetic or dermatological formulation such as alcohols, polyols, polymers, foam stabilizers, electrolytes, organic solvents or silicone derivatives. They can furthermore contain organic and/or inorganic oil-soluble, water-soluble and/or pigmentary UV lightscreen filters.

Advantageous preservatives within the meaning of the present invention are, for example, formaldehyde-cleaving agents (such as, for example, DMDM hydantoin, which is obtainable, for example, from Lonza under the trade name Glydant™), iodopropylbutyl carbamates (e.g. those obtainable from Lonza under the trade names Glydant-2000, Glycacil-L, Glycacil-S, and/or Dekaben LMB from Jan Dekker), parabens (i.e. alkyl p-hydroxybenzoates, such as methyl-, ethyl-, propyl- and/or butylparaben), phenoxy-ethanol, ethanol, benzoic acid and suchlike. Customarily, the preservation system according to the invention furthermore advantageously also comprises preservation aids, such as, for example, octoxyglycerol, Glycine soja etc.

Particularly advantageous preparations are furthermore obtained if antioxidants are employed as additives or active ingredients. According to the invention, the emulsions advantageously contain one or more antioxidants. As antioxidants which are favorable, but nevertheless to be used optionally, it is possible to use all antioxidants suitable or customary for cosmetic and/or dermatological applications.

Particularly advantageously within the meaning of the present invention, it is possible to use water-soluble antioxidants, such as, for example, vitamins, e.g. ascorbic acid and its derivatives.

Preferred antioxidants are furthermore vitamin E and its derivatives and also vitamin A and its derivatives.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or its derivatives are the antioxidant(s), it is advantageous to choose their respective concentrations from the range from 0.001 to 10% by weight, based on the total weight of the formulation.

If vitamin A or vitamin A derivatives, or carotenes or their derivatives are the antioxidant(s), it is advantageous to choose their respective concentrations from the range from 0.001 to 10% by weight, based on the total weight of the formulation.

It is particularly advantageous if the preparations according to the present invention contain cosmetic or dermatological active ingredients, preferred active ingredients being antioxidants which can protect the skin from oxidative stress.

Further advantageous active ingredients within the meaning of the present invention are natural active ingredients and/or their derivatives, such as, for example, alpha-lipoic acid, phytoene, D-biotin, coenzyme Q10, alpha-glucosylrutin, carnitine, carnosine, natural and/or synthetic isoflavonoids, creatine, taurine and/or β-alanine.

Formulations according to the invention which contain, for example, known antiwrinkle active ingredients such as flavone glycosides (in particular α-glycosylrutin), coenzyme Q10, vitamin E and/or derivatives and the like are particularly advantageously suitable for the prophylaxis and treatment of cosmetic or dermatological skin changes, such as occur, for example, on ageing of the skin (such as, for example, dryness, roughness and formation of dryness lines, itching, reduced refatting (e.g. after washing), visible vasodilatation (teleangiectasies, cuperosis), slackness and formation of wrinkles and lines, local hyper-, hypo- and malpigmentation (e.g. age spots), increased susceptibility to mechanical stress (e.g. fissurability) and the like). Furthermore, they are advantageously suitable against the syndrome of dry or rough skin.

The water-in-silicone emulsions according to the invention can be composed as customary cosmetic and/or dermatological preparations and serve for cosmetic or dermatological light protection, furthermore for the treatment, care and cleansing of the skin and/or of the hair and as a make-up product in decorative cosmetics.

According to their structure, water-in silicone-emulsions within the meaning of the present invention can, for example, be used as a skin cream, cleansing milk, day or night cream etc. It is optionally possible and advantageous to use the compositions according to the invention as a base for pharmaceutical formulations.

For application, the water-in-silicone emulsions are applied to the skin and/or the hair in adequate amount in the manner customary for cosmetics.

The use of starch and/or starch derivatives for the stabilization of water-in-silicone emulsions is also novel.

Furthermore, the use of water-in-silicone emulsions as a cosmetic and/or dermatological preparation is novel.

The use of water-in-silicone emulsions as a cosmetic and/or dermatological cream or lotion is furthermore novel.

Not least, the use of water-in-silicone emulsions as a lotion or cream for skincare, moisturizing the skin and/or smoothing the skin is novel.

The following examples are intended to illustrate the present invention without restricting it. All quantitative data, proportions and percentages are, if not stated otherwise, based on the weight and the total amount or on the total weight of the preparations.

| | 1 | 2 | 3 | 4 | 5 |
|---|---|---|---|---|---|
| Laurylmethicone | 3 | 2 | | | 0.5 |
| copolyol | | | | | |
| Cetyl dimethicone | | | 3 | 2 | 1.5 |
| copolyot | | | | | |
| Cera microcrystallina | | 1.5 | | 1.5 | 2.5 |
| Petrolatum | | | | | 1.5 |
| Cyclomethicone | 25 | 18 | 25 | 18 | 20 |
| Dimethicone | 2.5 | 2 | 2.5 | 2 | 1 |
| Phenyltrimethicone | 1 | 0.5 | 2.5 | 1 | |
| Dimethiconol | 0.5 | 1 | 0.5 | 1 | |
| Phenoxyethanol | 0.5 | 0.5 | 0.5 | 0.5 | |
| Parabens | | 0.2 | | 0.2 | 0.1 |
| Formaldehyde-cleaving | 0.3 | | 0.3 | | 0.1 |
| agents | | | | | |
| Iodopropynyl | | 0.1 | | 0.1 | 0.1 |
| butylcarbamate | | | | | |
| Magnesium sulfate | | 0.7 | 1.2 | | 0.5 |
| Sodium chloride | 1.5 | | | 2 | 0.8 |
| Propylene glycol | 3 | 5 | | 3 | |
| Butylene glycol | 4 | 6 | | 5 | |
| Glycerol | 6 | 8 | 10 | 12 | 20 |
| Alcohol, denat. | 3 | | 5 | | |
| Dimethylimidazolidone- | 1 | | 1 | | 0.5 |
| rice starch | | | | | |
| Aluminum starch | | 1 | | 2 | 0.5 |
| octenylsuccinate | | | | | |
| Tapioca starch | | 0.5 | | | 0.5 |
| Perfume | q.s. | q.s. | q.s. | q.s. | q.s. |
| Water | to 100 | to 100 | to 100 | to 100 | to 100 |

1.-13. (canceled).

14. A water-in-silicone emulsion comprising
(a) an aqueous phase,
(b) a lipid phase comprising at least 50% by weight of a silicone, based on the weight of the lipid phase,
(c) one or more emulsifiers, and
(d) at least one substance selected from starch and starch derivatives.

15. The emulsion of claim 14, wherein the emulsion comprises from 0.1% to 20% by weight of component (d).

16. The emulsion of claim 15, wherein the emulsion comprises at least about 0.5% by weight of component (d).

17. The emulsion of claim 16, wherein the emulsion comprises up to 10% by weight of component (d).

18. The emulsion of claim 14, wherein component (d) comprises at least one of dimethylimidazolidone rice starch, aluminum starch octenylsuccinate and tapioca.

19. The emulsion of claim 14, wherein component (c) comprises at least one of cetyldimethicone copolyol and laurylmethicone copolyol.

20. The emulsion of claim 15, wherein component (c) comprises cetyldimethicone copolyol.

21. The emulsion of claim 15, wherein component (c) comprises laurylmethicone copolyol.

22. The emulsion of claim 14, wherein component (c) comprises an alkyldimethicone copolyol and component (d) comprises dimethylimidazolidone rice starch.

23. The emulsion of claim 17, wherein component (c) comprises an alkyldimethicone copolyol and component (d) comprises dimethylimidazolidone rice starch.

24. The emulsion of claim 14, wherein the silicone of component (b) comprises at least one of dimethicone and cyclomethicone.

25. The emulsion of claim 15, wherein the silicone of component (b) comprises dimethicone.

26. The emulsion of claim 15, wherein the silicone of component (b) comprises cyclomethicone.

27. The emulsion of claim 15, wherein component (b) comprises up to 95% by weight of a silicone.

28. The emulsion of claim 27, wherein component (b) comprises at least 75% by weight of a silicone.

29. The emulsion of claim 14, wherein the ratio of component (a) and component (b) is from 65:35 to 80:20.

30. The emulsion of claim 29, wherein the ratio of component (a) and component (b) is at least 70:30.

31. The emulsion of claim 29, wherein the ratio of component (a) and component (b) is not higher than 75:25.

32. The emulsion of claim 14, wherein component (b) further comprises one or more polar oils.

33. The emulsion of claim 32, wherein the one or more polar oils comprise at least one of a lecithin and a fatty acid triglyceride.

34. The emulsion of claim 32, wherein component (b) comprises not more than 40% by weight of the one or more polar oils.

35. The emulsion of claim 14, wherein component (b) further comprises one or more nonpolar oils.

36. The emulsion of claim 35, wherein the one or more nonpolar oils comprise at least one of a hydrocarbon and a hydrocarbon wax.

37. The emulsion of claim 36, wherein component (b) comprises up to 50% by weight of the one or more nonpolar oils.

38. The emulsion of claim 14, wherein the emulsion further comprises one or more humectants.

39. The emulsion of claim 38, wherein the emulsion comprises from 5% to 25% by weight of the one or more humectants.

40. The emulsion of claim 39, wherein the emulsion comprises from 7.5% to 15% by weight of the one or more humectants.

41. The emulsion of claim 39, wherein the one or more humectants comprise at least one of glycerol, butylene glycol, propylene glycol, lactic acid, sodium lactate, glycine soja, ethylhexyloxyglycerol, pyrrolidone carboxylic acid, urea, hyaluronic acid and chitosan.

42. The emulsion of claim 38, wherein the one or more humectants comprise at least one of glycerol, butylene glycol and propylene glycol.

43. The emulsion of claim 14, wherein the emulsion comprises from 0.5% to 10% by weight of component (d), component (d) comprises at least one of dimethylimidazolidone rice starch, aluminum starch octenylsuccinate and tapioca, component (c) comprises at least one of cetyldimethicone copolyol and laurylmethicone copolyol, component (b) comprises from 75% to 95% by weight of a silicone, and the silicone of component (b) comprises at least one of dimethicone and cyclomethicone.

44. A cosmetic or dermatological cream or lotion which comprises the emulsion of claim 14.

45. A method of stabilizing a water-in-silicone emulsion, wherein the method comprises incorporating into the emulsion at least one of a starch and a starch derivative in an amount which is sufficient to stabilize the emulsion.

46. The method of claim 45, wherein the emulsion comprises an aqueous phase, a lipid phase comprising at least 50% by weight of a silicone, based on the weight of the lipid phase, and one or more water-in-silicone emulsifiers.

47. The method of claim 46, wherein 0.1% to 20% by weight of the at least one of a starch and a starch derivative are incorporated in the emulsion.

48. The method of claim 47, wherein the at least one of a starch and a starch derivative comprises one or more of dimethylimidazolidone rice starch, aluminum starch octenylsuccinate and tapioca.

49. A method of moisturizing or smoothing skin, wherein the method comprises applying onto at least parts of the skin a water-in-silicone emulsion which comprises
(a) an aqueous phase,
(b) a lipid phase comprising at least 50% by weight of a silicone, based on the weight of the lipid phase,
(c) one or more emulsifiers, and
(d) at least one substance selected from starch and starch derivatives.
